(19) United States
(12) Patent Application Publication
(10) Pub. No.: US 2002/0090718 A1
(43) Pub. Date: Jul. 11, 2002

(54) Device for composting organic material and the process for composting said organic material

(76) Inventors: Yona Chen, Ncs Ziona (IL); Yitzak Hadar, Rehovot (IL); Amir Toar, Moshav Mishmar Ayalon (IL)

Correspondence Address: BIRCH STEWART KOLASCH & BIRCH, PO BOX 747, FALLS CHURCH, VA, 22040-0747, US

(21) Appl. No.: 10/013,501

(22) Filed: Dec. 13, 2001

Related U.S. Application Data
(60) Provisional application No. 60/255,131, filed on Dec. 14, 2000.

(51) Int. Cl. C12M 1/02

(52) U.S. Cl. 435/290.3

(57) ABSTRACT

The present invention relates to a composter being an insulated container into which organic material may be loaded; said container comprising an outer plate of rigid material, an inner plate of material resistant to corrosion and a layer of heat insulating material being located between said two plates; the container being provided with loading and emptying means and suitable openings; and comprising means for rotating and homogenizing the organic material. The composter may be provided with a base and/or with wheels. The invention also relates to a process for composting organic material which comprises the steps of: periodic loading organic materials into the above composter; rotating and homogenizing the organic materials; and incubating the organic materials for a predetermined period.

The present invention relates to a device for composting organic material [hereinafter called “composter”] and to a process for composting said organic material.

Composting is defined as thermophilic aerobic decomposition of organic matter by a mixed microbial population conducted under controlled conditions. Composting is a form of organic waste stabilization which requires specific conditions, particularly moisture and aeration, to yield temperatures conducive to thermophilic microorganisms. Compost is the stabilized and sanitized product of composting which is beneficial to plant growth. During composting the heterogeneous organic input substrate undergones an initial, rapid stage of decomposition and is transformed into humus. Controlled environmental conditions distinguish composting from natural decomposition which often occurs in open dumps, manure heaps, or field soil. The temperature trend in most composting processes can be divided into four stages:

(1) Mesophihc stage—an initial phase lasting 1-2 days, during which mesophilic strains of microorganisms start to decompose readily degradable compounds of fresh natural organic matter, heat is given off and the temperature rises. The pH drops slightly as organic acids are produced.

(2) Thermophilic stage—the second stage, typically lasting 2-4 weeks, during which thermophilic microbial populations active above temperatures ranging from 45°-65° C. take over. If the temperature rises above 60° C., fungi become deactivated, and the reaction is continued by thermophilic bacteria. In this phase the more readily degradable substances such as sugars, fats, starch and proteins are rapidly consumed and most of the human and plant pathogens are destroyed; the pH increases and becomes alkaline as ammonia is liberated from the proteins. The reaction rate decreases as the more resistant materials are attacked; the composting process switches to stage 3.

(3) Cooling-down stage—6 to 10 weeks, as the temperature drops, thermophilic fungi reinvade the composted material. The first three stages of composting last from 8 to 12 weeks, in most systems (excluding those where forced aeration is employed), depending on the composted material and composting conditions.

(4) Stabilization and maturation stage—requires several months, where little heat is generated and the final pH is normally slightly alkaline. During this phase, mesophilic microorganisms as well as macrofauna colonize the compost. Intense competition for food takes place between the microorganisms, involving antagonism and the formation of antibiotics. Humification occurs in the residual organic matter to produce the stable composted product. (Stage 4 is typical for large scale composting, but is usually not employed in small volume composters).

Said known devices are described and claimed inter alia in the following specifications: PCT WO 95/31419 and WO 98/49122, EP 531 832 A1, U.S. Pat. Nos. 3,837,810, 4,042,219, 4,946,108, 5,457,031, 5,470,747, 5,890.664, 5,899,803 and 6,110,733.

There are various known composters which substantially consist of a static, non-insulated plastic device. In such a known device, the material cannot be homogenized efficiently and aerated, and it does not reach the desired temperature, due to heat loss to the environment. Moreover, said known composters are usually large and not easy to handle. All these problems, which hinder the composting process, have substantially been solved in the present invention.

It has thus been desirable to design a composter which would overcome the above drawbacks. Said composter should not be too large and it should be easy to handle, it should enable easy homogenizing due to the fact that the required temperature is easily achieved and maintained. Moreover, it should be easy to be manufactured and not be too expensive.

The present invention thus consists in a composter being an insulated container into which organic material may be loaded; said container comprising an outer plate of rigid material, an inner plate of material resistant to corrosion and a layer of heat insulating material being located between said two plates; the container being provided with loading and emptying means and suitable openings; and comprising means for rotating and homogenizing the organic material.

The heat insulating layer maintains the optimal internal temperature.

In a preferred embodiment of the present invention, said container has a cylindrical shape and the plates have the form of sleeves.

The outer sleeve is preferably made of galvanized steel having a thickness of about 1.5 mm. The inner sleeve is preferably made from a suitable plastic material, e.g. PVC, etc. The heat insulating material is preferably a layer of foamed polyurethane or of any suitable material, e.g. glass wool, etc. The distance between the two sleeves is suitably about 40 mm forming a space in which space the insulating layer is located.

The composter according to the present invention may comprise a base which positions the container.

The base advantageously consists of special steel profiles with two special bearings: a manual rotating handle, and a brake system provided with a locking means. The bearings insure smooth turning of the container, the manual rotating handle enables the transfer of the force, e.g. via sprockets and driving chains and the brake system with the locking means stops free rotation of the container.

Due to the heavy weight of the composter, it may be connected via the base to a power take-off system connected to a tractor. Alternatively, the base may comprise a motor, having the capacity to rotate the composter.

The entire composter may be provided with wheels which enable easy transfer of the composter to any required site.

The homogenizing means are advantageously located in the inner space of the container. Said homogenizing means are, for example, agitators, e.g. handle bars welded around the steel circumference of the container; buffles; etc. The loading and emptying means are, e.g. a hatch and the openings are, e.g. ventilating holes.

The composter according to the present invention is preferably small to medium, i.e. 200-4000 liter.

The process according to the present invention comprises the steps of: periodic loading organic materials into the composter according to the present invention; rotating and homogenizing the organic materials; and incubating the organic materials for a predetermined period.

The homogenizing and incubating of the organic material depends on the various parameters such as the volume, content etc. of the organic material.

The temperature of the process preferably varies between 50-65° C. The process for home utilization involves continuous filling. The rate of inputs and volume of the composter determines the time period until the composter should be emptied—in total about 6 to 10 weeks.

The process according to the present invention is suitably performed as follows:

In said process the container is preferably rotated 10 rotations, every 3-5 days (or as desired). After one week of composting it is possible to add another load of organic waste. After 4 to 6 additions, the material is left for 3 weeks of composting and then transferred for storage (maturation) or use.

The composters according to the present invention have several special advantages:

1. The composter is totally insulated to avoid heat loss during the process, resulting in process optimization, thereby achieving significant shortening of the process time.

2. Standardization of the parts gives high versatility in delivery of composters varying in their volumes (200-4000 L) and a short lead time,

3. Homogenizing the content of the composter by rotation, in order to achieve better mixing.

4. The composter is massively built to function for many years.

5. The composter can be mobilized on wheels and easily transferred from site to site.

6. The inner plastic sleeve is protected from corrosion and leachates.

7. The composter can be easily loaded and unloaded with the organic waste.

8. The flexibility of availability of sizes of composters allows versatile mode of operation using several composters in parallel and partial sequence.

9. The composting process, using this composter, is by far more effective than the currently available plug flow systems.

The invention will now be described and illustrated with reference to the accompanying drawings and to the examlples without being limited to them.

In said drawings:

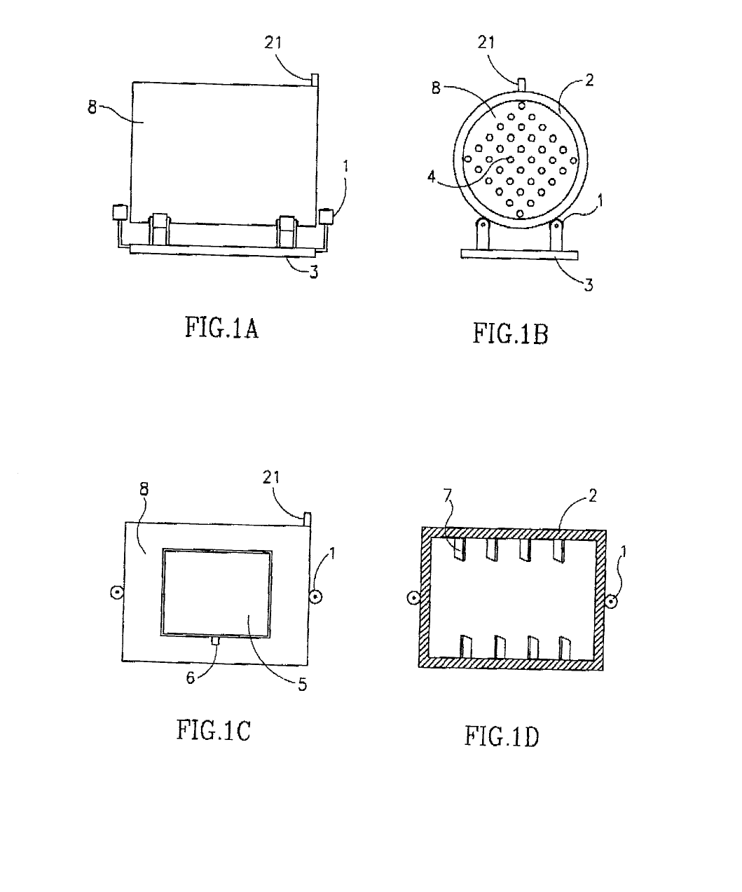

FIG. 1 shows various parts of a composter as described in Example 1. Said parts show:

FIG. 1A: a front elevator;

FIG. 1B: a side elevator;

FIG. 1C: a plan; and

FIG. 1D: a section.

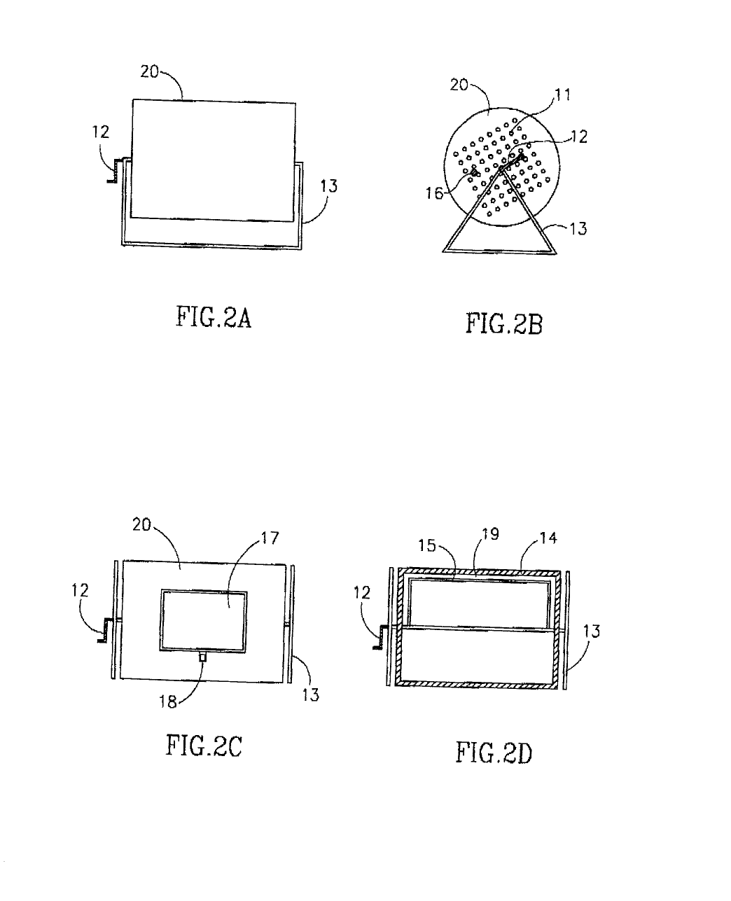

FIG. 2 shows various parts of a composter as described in Example 2. Said parts show:

FIG. 2A: a front elevator;

FIG. 2B: a side elevator;

FIG. 2C: a plan; and

FIG. 2D: a section.

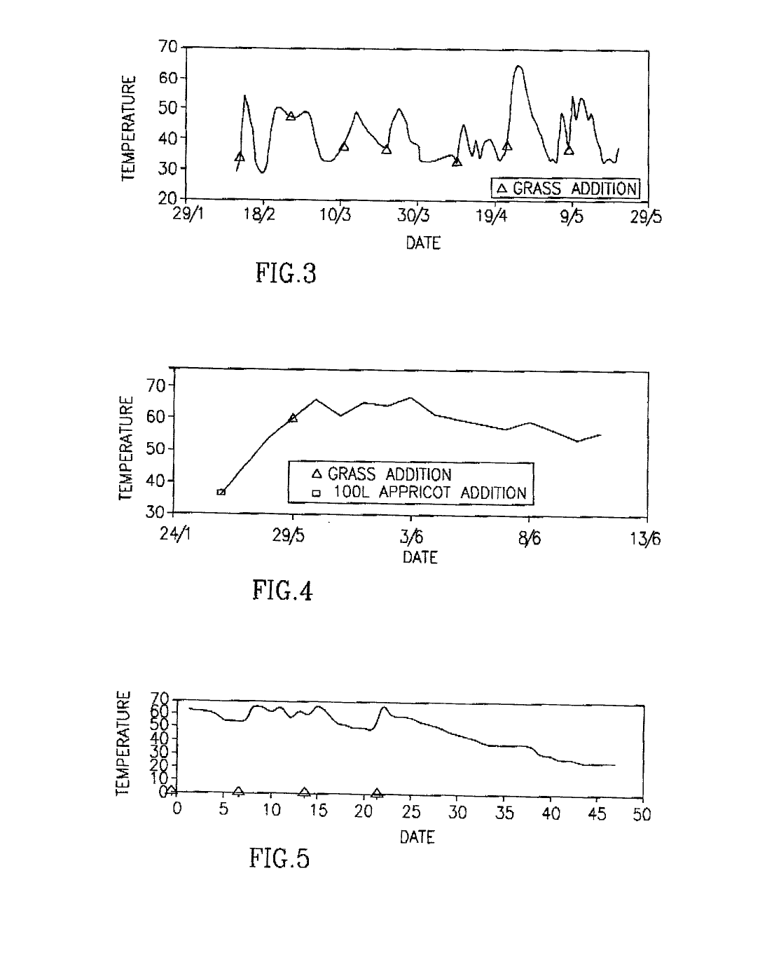

FIGS. 3 to 6 show the results of the test described in Examples 3 to 6.

### EXAMPLE 1

### 200 Liter Insulated Composter

The 200 or 500 liter composter is designed for household backyard composting. It consists of a container 8 and a base 3. The container 8 is cylindrical shaped and the outer sleeve is produced from 1.5 mm. galvanized steel rolled into the final shape and size. Between the two sleeves is a 40 mm heat insulation layer 2 made of foamed polyurethane. The container has a loading and emptying hatch 5 and ventilating holes 4. The base 3 is built to carry the full load with special okollon wheels 1 and to enable thorough mixing. Mixing can be performed by one person and is facilitated by specific handle bars 21 welded around the steel circumference of the container. The inner space includes (i) special buffles 7 to enable homogenizing of the raw materials and the organic matter during the composting process; and (ii) an insulation layer 2 that keeps an optimal internal temperature. A 200 liter composter is illustrated in FIG. 1.

### EXAMPLE 2

### (2) 1000-4000 Liter Insulated Composter

This composter is designed for institutional use. The device consists of a container 20 and a base 13. The size of the container can range from 1000 liter up to 4000 liter, depending on consumer demand. A 1000 liter composter is illustrated in FIG. 2.

The base 13 consists of special steel profiles with two special bearings to ensure smooth turning of the container; a manual rotating handle 12 transfering the force via sprockets and driving chains; and a brake system with a locking pin 16 to stop free rotation of the container. Two other optional rotating technologies have been developed and can be included in the apparatus; (i) power take-off from a tractor; and (ii) a small electric or gasoline motor.

The container is cylindrical shaped; the inner sleeve 19 is produced from special plastic material; the outer sleeve 20 is produced from 1.5 mm. galvanized steel rolled into the final shape and size. Between the two sleeves is a 40 mm heat insulation layer 14 made of foamed polyurethane. The container has a loading and emptying hatch 17 and ventilating holes 11 The outer rigid steel envelope 20 is built to carry the full load. The inner space includes special agitators 15 to enable homogenizing of the raw materials and the organic matter during the composting process and the insulation layer 14 keeps the right internal temperature.

The composter can be built with wheels, according to customer demand, to enable easy transfer of the device to different sites.

### EXAMPLE 3

Composting of household waste in a 200 liter composter as illustrated in Example 1 and FIG. 1. A family of four persons used a composter 200 for the recycling through composting of the organic household waste, comprising kitchen waste plus garden waste for a period of 3 months. Grass clippings were added every two weeks, and kitchen waste was added daily. Temperature was measured throughout the composting process and the composter apparatus was turned by rolling every 3 days. FIG. 3 shows the temperature profile during this period of time, Temperatures above the ambient are indicative of microbial decomposition of the organic matter typical for the composting process. The temperature rises as a response to the grass clippings added and the composter maintains the high temperature due to the insulation. Since the process took place under optimal conditions (temperature and mixing) at the end of the 3-month period, the decomposed waste (compost) was ready for use as a soil conditioner in the family garden.

### EXAMPLE 4

The same composter as used in Example 3 was used by the same family, as a test case. About 30 kg of apricots were added at the beginning of the experiment (yield of the family garden unsuitable for consumption), followed by daily additions of kitchen waste and periodical additions of grass clippings. The temperature profile of the process is shown in FIG. 4.

### EXAMPLE 5

The same composter as used in Example 3 was used to compost grass clippings. The raw material was added once a week, for 4 weeks. The apparatus was mixed every 3 days. The temperature increased to 65° C. within a day and was kept at a level of above 50° C. until day 25. The compost produced was suitable for growth of cucumbers, as a container medium (FIG. 5).

### EXAMPLE 6

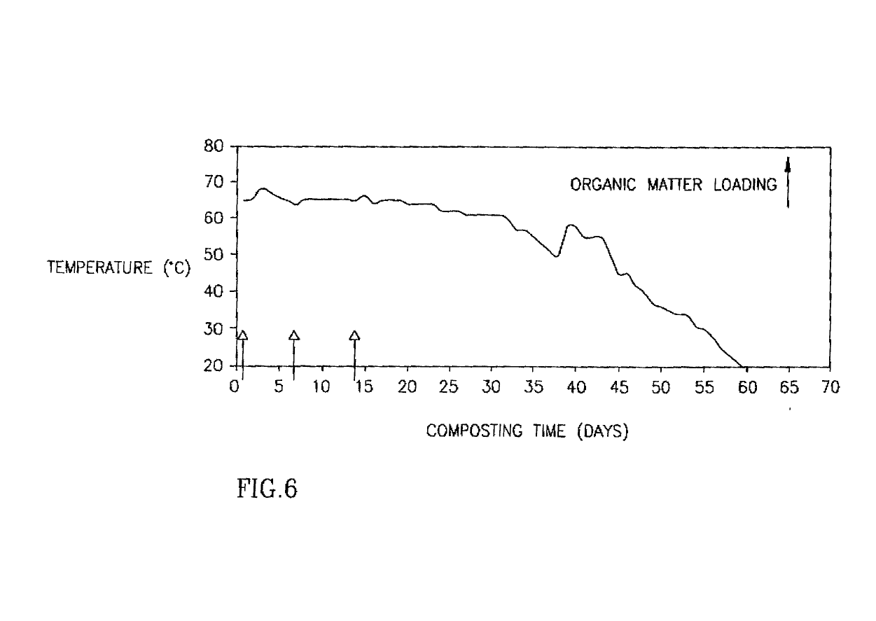

The composter as illustrated in Example 2 and FIG. 2 was filled with grass clippings. The temperature increased within one day to 65° C. and the volume decreased to 500 L within 7 days. At that time, the composter was refilled and the process was continued. At day 14, the composter was filled for the 3 time. The apparatus was mixed every 3 days for the first month. As a result of the mixing and the insulation, the temperature was kept at 65° C. for 45 days. At this time, the microbial activity declined due to complete utilization of the available organic matter, and thus a decrease of the temperature was observed. The composting of 2500 liters of raw material yielded 250 liters of mature compost. This significant decrease of waste volume was achieved, as well as high quality compost for use as a soil conditioner. The temperature profile is shown in FIG. 6.

1. A composter being an insulated container into which organic material may be loaded; said container comprising an outer plate of rigid material, an inner plate of material resistant to corrosion and a layer of heat insulating material being located between said two plates; the container being provided with loading and emptying means and suitable openings; and comprising means for rotating and homogenizing the organic material.

2. A composter according to claim 1, wherein the container has a cylindrical shape and the plates have the form of sleeves.

3. A composter according to claim 2, wherein the outer sleeve is made of galvanized steel having a thickness of about 1.5 mm.

4. A composter according to claim 2, wherein the inner sleeve is made from a suitable plastic material, e.g. PVC.

5. A composter according to claim 2, wherein the heating insulating material is a layer of foamed polyurethane.

6. A composter according to claim 2, wherein the space between the two sleeves is about 40 mm.

7. A composter according to claim 1, comprising a base.

8. A composter according to claim 7, wherein the base consists of special steel profiles with two special bearings; a manual rotating handle, and a brake system provided with a locking means.

9. A composter according to claim 7, wherein the composter is connected via the base to a power take-off system connected to a tractor.

10. A composter according to claim 7, wherein the base comprises a motor having the capacity to rotate the composter.

11. A composter according to claim 1, being provided with wheels.

12. A composter according to claim 1, wherein the homogenizing means are loaded within the inner space.

13. A composter according to claim 1, wherein the homogenizing means are, eg. handle bars welded around the steel circumference of the container; buffles etc.

14. A composter according to claim 1, wherein the loading and emptying means are, e.g. a hatch and the openings are e.g. ventilating holes.

15. A process for composting organic material which comprises the steps of periodic loading organic materials into the composter according to claim 1 rotating and homogenizing the organic materials; and incubating the organic materials for a predetermined period.

16. A process according to claim 15, wherein the performance temperature is 50-65° C.

17. A process according to claim 15, wherein the home utilization is performed with continuous filling and the time period for the performance is about 6 to 10 weeks.
